(19) United States
(12) Patent Application Publication
(10) Pub. No.: US 2014/0356302 A1
(43) Pub. Date: Dec. 4, 2014

(54) Buoyant Cosmetic Gel Formulation

(71) Applicant: Doris Susie Yuen, Oxnard, CA (US)

(72) Inventor: Doris Susie Yuen, Oxnard, CA (US)

(73) Assignee: SPATZ LABORATORIES, Oxnard, CA (US)

(21) Appl. No.: 14/294,656

(22) Filed: Jun. 3, 2014

Related U.S. Application Data
(60) Provisional application No. 61/830,707, filed on Jun. 4, 2013.

(51) Int. Cl. A61K 8/73; A61Q 1/02; A61K 8/04

(52) U.S. Cl. CPC A61K 8/73; A61K 8/042; A61Q 1/02; A61K 2800/43
USPC 424/63

(57) ABSTRACT

A buoyant cosmetic gel formulation with a high moisture content that increases the longevity and facilitates the application of the formulation on a user's skin through various means of application. The buoyant cosmetic gel formulation contains a volatile liquid, a polysaccharide as a film forming and structuring agent, a humectant, effect pigments, and an anhydrous phase. The formulation suspends the effect pigments in a manner in an aqueous phase formed by the polysaccharide and the volatile liquid. The formulation is externally applied on a user's skin and is perceived as soft and light weight.

The current application claims a priority to the U.S. Provisional Patent application Ser. No. 61/830,707 filed on Jun. 4, 2013.

### FIELD OF THE INVENTION

The present invention relates generally to a cosmetic formulation. More specifically, the present invention relates to a water based buoyant gel cosmetic formulation that is applied externally to a user's skin.

### BACKGROUND OF THE INVENTION

The majority of color cosmetics in the marketplace are anhydrous. Many of them are dry powders either in loose or pressed form, molded lipsticks and eye pencils that are oil and wax based, or eyeliners, mascaras, or lip glosses in a cream or liquid form. There are few color cosmetics that have a high water content. Moreover, none of the color cosmetics that have a high water content are in a buoyant gel form and has the attributes of long wear, versatility by use of brush or finger application, does not dust or flake, feels extremely lightweight on skin, and delivers a luminous sheen and clarity on skin. Because of this, there is a gap in the cosmetic marketplace that can be fulfilled to satisfy consumers for a need of a lightweight, comfortable feeling product that is cooling and refreshing upon application, yet has the benefit of long wear with no dusting or flaking. Categories that this cosmetic product form include but are not limited to products for the eye, face, lip, lash, and nail.

It is therefore an object of the present invention to introduce a composition for external use on skin that is aqueous based, contains one or more polysaccharide(s) as film former(s) and structuring agent(s), contain effect pigments, and is structurally soft and buoyant, but does not flow or slide in the component that it is contained in and does not break upon mechanical force of the finger or brush for cosmetic application.

### DETAIL DESCRIPTIONS OF THE INVENTION

The present invention is a buoyant cosmetic gel formulation that is formulated with a high moisture content that increases its longevity and facilitates its application on a user's skin through various means of application. The buoyant cosmetic gel formulation is light weight and can be comfortably worn for an extended period of time. In the current embodiment of the present invention, the buoyant cosmetic gel formulation comprises a volatile liquid, at least one polysaccharide, at least one effect pigment, a humectant, and an anhydrous phase. The volatile liquid provides moisture to the buoyant cosmetic gel formulation and forms the aqueous phase with that at least one polysaccharide. The at least one polysaccharide functions as a film former and as a structuring agent and when combined with the volatile liquid. The at least one effect pigment is a composite of various compounds that function as a colorants, opacifying agents, and/or lustrous agents. The humectant is a hygroscopic substance that prevents the loss of moisture in the buoyant cosmetic gel formulation. The anhydrous phase is immiscible with the aqueous phase and can impart water resistances to the buoyant cosmetic gel formulation.

TABLE 1
Buoyant Cosmetic Gel Formulation

| Compounds | wt % |
|---|---|
| Volatile Liquid | 30.0-70.0 wt % |
| At Least One Polysaccharide | 0.01-10.0 wt % |
| At Least One Effect Pigment | 10.0-50.0 wt % |
| Humectant | 1.0-30.0 wt % |
| Anhydrous Phase | 1.0-30.0 wt % |

The volatile liquid is the aqueous substance that provides moisture to the buoyant cosmetic gel formulation. The volatile liquid is combined with the at least one polysaccharide to form the aqueous phase. The aqueous phase has increased viscosity compared to the volatile liquid as a result of the combination with the at least one polysaccharide. The aqueous phase functions as the carrier for the at least one pigment. The humectant is soluble in the volatile liquid. In the current embodiment of the present invention, the volatile liquid in the formulation is found ranging between 30.0 to 70.0 wt % of the formulation. The quantity range of the volatile liquid provides the buoyant cosmetic gel formulation with its high moisture content. The volatile liquid has similar volatility to water and as a result has a vapor pressure that is near 3.169 kPa at 25° C. The volatile liquid can be provided as a mixture of aqueous volatiles provided the combined vapor pressure of the liquids is near that of water. In the preferred embodiment of the present invention, the volatile liquid is water. Water is selected as the preferred volatile liquid for its solvent properties as well as its low risk of causing irritation to a user's skin.

TABLE 2
Polysaccharide Group

| Polysaccharides | Function |
|---|---|
| Gellan Gum | Viscosity Increasing Agent; Film Forming |
| | Agent; |
| Xanthan Gum | Viscosity Increasing Agent; Thickener; |
| | Emulsion Stabilizer; |
| Agar | Thickener; Gelling Agent; |
| Carrageenan | Gelling Agent; Thickener; Emulsion |
| | Stabilizer; |
| Locust Bean Gum | Viscosity Increasing Agent; Thickener; |
| | Film Forming Agent; |
| Acia Senegal Gum | Film Forming Agent; |
| Guar Gum | Viscosity Increasing Agent; Thickener; |
| | Film Forming Agent; |
| Konjac Mannan Gum | Thickener; Emulsion Stabilizer; |

The at least one polysaccharide is provided as a film forming agent and structuring agent in the formulation. The at least one polysaccharide is combined with the volatile liquid to form the aqueous phase. The at least one polysaccharide functions as a film forming agent by imparting the buoyant cosmetic gel formulation with properties that enabling it to form a pliable, cohesive, and continuous covering when applied over a user's skin. The at least one polysaccharide functions as a structuring agent functioning as a gelling, thickening, and emulsion stabilizing agent providing the buoyant cosmetic gel formulation with its increased viscosity. In the current embodiment of the present invention, the at least one polysaccharide is found ranging between 0.01 to 10.0 wt % of the formulation. The quantity range of the at least one polysaccharide permits the buoyant cosmetic gel formulation to have varying viscosities enabling its incorporation into a plurality of different cosmetic products. It should be noted that the quantity range of the at least one polysaccharide is the aggregate wt % of all polysaccharides incorporated into the formulation.

The at least one polysaccharide in the buoyant cosmetic gel formulation is selected from a group of polysaccharides consisting of gellan gum, xanthan gum, agar, carrageenan, locust bean gum, acacia senegal gum, guar gum, and konjac mannan gum. Gellan gum is a tetrasaccharide consisting of two residues of D-glucose and one of each residues of L-rhamnose and D-glucuronic acid. Xanthan Gum is a pentasaccharide comprising glucose, mannose, and glucuronic acid in the molar ratio 2.0:2.0:1.0. Agar is a polysaccharide comprising agrose units. Carrageenan is a linear sulfated polysaccharide with a high-molecular-weight made up of repeating galactose units and 3,6 anhydrogalactose units. Locust Bean Gum and Guar Gum contain the polysaccharide Galactomannan which is a high-molecular-weight hydrocolloidal polysaccharides comprising galactose and mannose units. Acacia Senegal Gum, a grade of Gum Arabic, is a biopolymer comprising arabinose and galactose monosaccharides. Konjac Mannan Gum contains the polysaccharide Glucomannan, a straight-chain polymer with a small amount of branching comprising β-(1→4)-linked D-mannose and D-glucose in a ratio of 1.6:1.

In the preferred embodiment of the present invention, the at least one polysaccharide is carrageenan. Carrageenan is a linear sulfated polysaccharide with a high-molecular-weight made up of repeating galactose units and 3,6 anhydrogalactose units. Carrageenan is selected as the preferred polysaccharide due to its inherent gelling, thickening, and emulsion stabilizing properties permitting it to function as a film forming and structuring agent in the buoyant cosmetic gel formulation.

While the at least one polysaccharide is selected from the aforementioned group of polysaccharides, it should be noted that the at least one polysaccharide can be accomplished by polysaccharides that include but are not limited to gellan gum, xanthan gum, agar, carrageenan, locust bean gum, acacia senegal gum, guar gum, welan, karaya gum, okra gum, aloe gum, gum targacanth, gum ghatti, quinceseed gum, psyllium, starch arabinogalactan, and konjac mannan gum as well as any combination thereof.

TABLE 3
Effect Pigments

| Groups | Compounds |
|---|---|
| Particulate Substrate | Mica; Synthetic Mica; Silica; |
| | Glass Flakes; Alumina Powder; |
| Metal Oxides | Titanium Dioxide; Iron Oxide; |
| | Chromium Oxide; |
| Colorant Compounds | Aluminum Powder; Bismuth Oxychloride; |
| | Borosilicates; Ferric Ferrocyanide; |
| | Ultramarines; Manganese Violet; |
| | Carmine; Organic Pigments; |

The at least one effect pigment is a composite of various compounds that provides color, iridescence, luster, and/or tactile properties to the buoyant cosmetic gel formulation. The at least one effect pigment is homogenously suspended throughout the aqueous phase. In the preferred embodiment of the present invention, the at least one pigment is found ranging between 10.0 to 50.0 wt % of the formulation. The quantity range of the at least one pigment provides the buoyant cosmetic gel the ability to vary intensity and appearance of the colorants, opacifying agents, and/or lustrous agents. The at least one effect pigment is a composite of various compounds that include a particulate substrate, a metal oxide compound, and a colorant compound. The particulate substrate has opacifying, tactile, and lustrous properties and functions in combination with the metal oxide compound and colorant compound. The metal oxide compounds can be colored or uncolored and impart lustrous and iridescent effects to the formulation. The colorant compounds are pigments that can be accomplished by a plurality of compounds. It should be noted that the quantity range of the at least one effect pigment is provided as the aggregate wt % of the composite of various compounds making up the effect pigment.

The particulate substrate is provided in combination with the colorant compound and/or the metal oxide compound. The particulate substrate is shaped dependent on desired effect sought for the formulation. The particulate substrate can be coated or encapsulated by a metal oxide compound and colorant compound as well as provided separately. In an embodiment of the invention, the at least one effect pigment solely comprise the particulate substrate. In the current embodiment of the present invention, the particulate substrate is selected from a group consisting of mica, synthetic mica, silica, glass flakes, and alumina. While the particulate substrate is selected from the aforementioned group of particulate substrates, it should be noted that in an embodiment of the invention, the particulate substrate is not limited to the aforementioned group of particulate substrates and can additionally include recombination thereof to improve the aesthetic effects of the formulation.

The metal oxide compounds are colored or uncolored compounds that impart lustrous and iridescent effects to the formulation. The metal oxide compounds are provided in combination with the particulate substrate and can either coat or encapsulate the particulate substrate. The colored variations of the metal oxide compounds can be accomplished by combining a metal oxide compound with a colorant compound. The metal oxide compounds can be provided separately from the particulate substrate and function as the principle compound of the at least one effect pigment. In the current embodiment of the present invention, a metal oxide compound is selected from a group consisting of titanium dioxide, iron oxide, and chromium oxide. It should be noted that in an embodiment of the invention, the metal oxide compound would not be limited to the aforementioned group of metal oxides and could be accomplished by any suitable metal oxide compound. Suitable metal oxide compounds include but are not limited to aluminium oxide, silicon oxide, iron oxide, tin oxide, cerium oxide, zinc oxide, zirconium oxide, chromium oxide, titanium oxide, and titanium dioxide as well as any combination thereof.

The colorant compounds are primarily provided to enhance pigmentation of the buoyant cosmetic formulation. The colorant compounds are provided in combination with the particulate substrate and/or with the metal oxide compounds. Dependent upon the particular needs of a cosmetic product in which the buoyant cosmetic gel formulation is found, the colorant compounds can be provided as the sole component of the at least one effect pigment. In the current embodiment of the present invention, the colorant compounds are selected from a group consisting of aluminum powder, bismuth oxychloride, borosilicates, ferric ferrocyanide, ultramarines, manganese violet, carmine, and organic pigments. It should be noted that colorant compounds can be accomplished by combinations of the compounds in the aforementioned group of colorant compounds. Furthermore it should be noted that in an embodiment of the invention, the colorant compound may be accomplished by any suitable colorant compound, including compounds not in the aforementioned group. Suitable colorant compounds would be dependent on the particular needs of a cosmetic product in which the buoyant cosmetic gel formulation is found.

TABLE 4
Humectants

| Class | Compounds |
|---|---|
| Glycols | Ethylene Glycol; Diethylene Glycol; |
| | Triethylene Glycol; Propylene Glycol; |
| | Dipropylene Glycol; 1,3-butylene Glycol; |
| | 1,4-Butylene Glycol; |
| Glycerin | Glycerin; Diglycerin; |
| Sugar Alcohols | Sorbitol; Mannitol; Xylitol; Maltitol; |

The humectant is a hygroscopic substance that prevents the loss of moisture in the buoyant cosmetic gel formulation. The humectant is soluble in the volatile liquid and would be provided in quantities that would be unlikely to degrade the at least one polysaccharide compound in the aqueous phase. In the current embodiment of the present invention the humectant is provided ranging between 1.0 to 30.0 wt % of the formulation. The quantity range of the humectant is provided to accommodate the particular needs of a cosmetic product in which the buoyant cosmetic gel formulation is found. The humectant is a polyol, an organic alcohol containing multiple hydroxyl group (—OH) enabling its solubility in the volatile liquid. The humectant utilized in the formulation is selected from a group of polyols consisting of glycols, glycerin, and sugar alcohols. Additionally, the humectant can be accomplished by any combination of compounds in the aforementioned group.

The glycols utilized as a humectant may be accomplished by any a suitable glycol such as but not limited to ethylene glycol, diethylene glycol, triethylene glycol, propylene glycol, dipropylene glycol, 1,3-butylene glycol, and 1,4-butylene glycol as well as any combination thereof. Glycerin and Diglycerin may be utilized the humectant. The sugar alcohols can be accomplished by any suitable sugar alcohol such as but not limited to sorbitol, mannitol, xylitol, and maltitol as well as any combination thereof.

In an embodiment of the present invention, the humectant may be accomplished by any suitable polyol. Suitable polyols include but are not limited to fructose, glucamine, glucose, glucose glutamate, glucuronic acid, glycerin, 1,2,6-hexanetriol, hydroxystearyl methylglucamine, inositol, lactose, malitol, mannitol, methyl gluceth-10, methyl gluceth-20, methyl glucose dioleate, methyl glucose sesquicaprylate/sesquicaprate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, thioglycerin, xylitol, and combinations thereof.

TABLE 5
Anhydrous Phase

| Class | Compounds |
|---|---|
| Silicone Compounds | Silicone Chain Polymers; Cyclic Silicone |
| | Compounds; Acrylic Silicone Polymers; |
| | Polymethylsiloxanes; Methicone; |
| | Dimethicone; |
| Oils | Synthetic Oils; Animal derived Oils; |
| | Vegetable Derived Oils; Mineral Oils |
| | Paraffin Oils; Silicone Oils; |
| Esters | Fatty Acid Esters; Isopropyl Alcohol |
| | Esters; Phytostearyl Esters; Synthetic |
| | Esters; Hydroxyl Esters; Neopentanoic |
| | Esters; Polyol Esters; Pentaerythritol |
| | Esters; Diol Dimer Esters; Diacid Dimer |
| | Esters; |
| Hydrocarbons | Linear Hydrocarbons; Branched |
| | Hydrocarbons; |
| Polymers | Organic Polymers; Inorganic Polymers; |
| | Acrylic Polymers; Synthetic Polymers; |
| Emulsifiers | Albumin; Alginates; Casein; Desoxycholic |
| | acid; Egg Yolk; Lecithin; Monosodium |
| | phosphate; Monostearate; Soaps; |
| | Taurocholic acid; |
| Waxes | Carnauba Wax; Beeswax; Ceresine Wax; |
| | Petroleum Wax; Ozokerite Wax; |
| | Microcrystalline Wax; |

The anhydrous phase is immiscible with the aqueous phase and can impart water resistances to the buoyant cosmetic gel formulation. The anhydrous phase is provided as any collection of compounds that are immiscible in the aqueous phase. The anhydrous phase can impart a plurality of properties to the buoyant cosmetic gel formulation but would be dependent on the particular needs of a cosmetic product in which the buoyant cosmetic gel formulation is found. In an embodiment of the invention, the anhydrous phase can be provided to impart water resistance to the buoyant cosmetic gel formulation increasing the amount of time a cosmetic product can be worn. In the current embodiment of the present invention, the anhydrous phase is found ranging between 1.0 to 30.0 wt % of the formulation. The quantity range of the anhydrous phase is provided to accommodate the particular needs of a cosmetic product in which the buoyant cosmetic gel formulation is found. The compounds in the anhydrous phase are selected from a group consisting of silicone compounds, oils, esters, hydrocarbons, polymers, emulsifying compounds, and waxes. It should be noted that the anhydrous phase may be accomplished by any combination of compounds from the aforementioned group. Furthermore, it should be noted that in an embodiment of the invention, the anhydrous phase may be accomplished by any suitable compound, including compounds absent from the aforementioned group, dependent on the particular needs of the cosmetic product.

The Silicone compounds utilized as in the anhydrous phase may include any suitable silicone compound utilized for cosmetic application. Suitable silicone compounds include but are not limited to silicone chain polymers, cyclic silicone compounds, acrylic silicone polymers, polymethylsiloxanes (PDMS), methicone, dimethicone, and combinations thereof.

The oils utilized as in the anhydrous phase may include any suitable oil utilized for cosmetic application. Suitable oils include but are not limited to synthetic oils, animal or vegetable derived oils, mineral oils, paraffin oils, silicone oils, and combinations thereof.

The esters utilized as in the anhydrous phase may include any suitable ester utilized for cosmetic application. Suitable esters include but are not limited Fatty acid esters, isopropyl alcohol esters, phytostearyl esters, synthetic esters, hydroxyl esters, neopentanoic esters, polyol esters, pentaerythritol esters, diol dimer esters, and diacid dimer esters, and combinations thereof.

The hydrocarbons utilized as in the anhydrous phase may include any suitable hydrocarbon utilized for cosmetic application. Suitable hydrocarbons include but are not limited to linear hydrocarbons, branched hydrocarbons, and hydrocarbons with cyclic functional groups as well as any combination thereof

The polymers utilized as in the anhydrous phase may include any suitable polymer utilized for cosmetic application. Suitable polymers include but are not limited to organic polymers, inorganic polymers, acrylic polymers, synthetic polymers, and combinations thereof.

The emulsifier utilized as in the anhydrous phase may include any suitable emulsifier utilized for cosmetic application. Suitable emulsifying compounds include but are not limited to Albumin, Alginates, Casein, Desoxycholic acid, Egg Yolk, Lecithin, Monosodium phosphate, Monostearate, Soaps, Taurocholic acid, and combinations thereof.

The waxes utilized as in the anhydrous phase may include any suitable wax utilized for cosmetic application. Suitable waxes include but are not limited to carnauba wax, beeswax, ceresine wax, petroleum wax, ozokerite wax, microcrystalline wax, and combinations thereof.

The invention is a buoyant gel that is applied externally on a user's skin decorative cosmetic purposes. The formulation contains one or more polysaccharide(s) as film former(s) and structuring agent(s) and effect pigments that do not flow or slide within the gel. The buoyant gel does not break upon mechanical force of the finger or brush for cosmetic application.

The invention comprises a volatile liquid, a polysaccharide, a humectant, an effect pigment, and an anhydrous phase. The volatile in this composition is either water or other volatile liquids that are aqueous based that are similar in volatility to that of water. The polysaccharides in this composition include but are not limited to gellan gum, xanthan gum, agar, carrageenan, locust bean gum, Acacia Senegal gum, guar gum, and konjac mannan gum. The effect pigments in this composition include but are not limited to colored and non-colored effect pigments based on mica, synthetic mica, alumina, aluminum powder, bismuth oxychloride, borosilicates, and glitters. The effect pigments may contain titanium dioxide, iron oxides, chromium oxides, ferric ferrocyanide, ultramarines, manganese violet, carmine, organic pigments, and dyes. The humectants in this composition include but are not limited to glycols, glycerin, and polyols. The anhydrous phase in this composition refers to the anhydrous components excluding the effect pigments. The components of the anhydrous phase include but are not limited to silicones, oils, esters, hydrocarbons, polymers, emulsifiers, and waxes. The cosmetic composition contains water or a mixture of aqueous volatiles and one or more polysaccharide(s) as the film former(s) to provide a long wearing formula. The polysaccharide(s) in the composition serve as structuring agent.

The buoyant gel structure suspends a substantial amount of effect pigments to deliver payout for decorative purposes. Gel structure is contained in a pot and is soft and bouncy to the touch but does not break upon mechanical force of the finger or brush for cosmetic application

The invention contains a substantial amount of powder to deliver payout but does not dust or flake upon application like a traditional pressed or loose powder cosmetic product would. Resulting application of the formulation feels extremely lightweight and comfortable as if no product is on the skin due to its composition of the product on the skin after the volatile has volatilized. The formulation is a water based cosmetic formulation that delivers luminous sheen and clarity on skin

The buoyant cosmetic gel formulation is aqueous based and contains one or more polysaccharide(s) as film former(s) and structuring agent(s), effect pigments, and is structurally soft and buoyant. The effect pigments do not flow or slide in the component that it is contained in and does not break upon mechanical force of the finger or brush for cosmetic application. The buoyant cosmetic gel formulation is for external use on the skin for decorative purposes and fulfills a niche in the cosmetic marketplace which is currently lacking cosmetics of this product form.

Although the invention has been explained in relation to its preferred embodiment, it is to be understood that many other possible modifications and variations can be made without departing from the spirit and scope of the invention as hereinafter claimed.

What is claimed is:

1. A buoyant cosmetic gel formulation comprising:
a volatile liquid;
at least one polysaccharide;
at least one effect pigment;
a humectant;
an anhydrous phase;
the volatile liquid being found ranging between 30.0 to 70.0 wt % of the formulation;
the at least one polysaccharide being found ranging between 0.01 to 10.0 wt % of the formulation;
the at least one effect pigment being found ranging between 10.0 to 50.0 wt % of the formulation;
the humectants being found ranging between 1.0 to 30.0 wt % of the formulation; and
the anhydrous phase being found ranging between 1.0 to 30.0 wt % of the formulation.

2. The buoyant cosmetic gel formulation as claimed in claim 1, wherein the volatile liquid is an aqueous liquid with similar volatility to water.

3. The buoyant cosmetic gel formulation as claimed in claim 2, wherein the volatile liquid is water.

4. The at least one polysaccharide as claimed in claim 1 is selected from a group consisting of gellan gum, xanthan gum, agar, carrageenans, locust bean gum, acacia senegal gum, guar gum, and konjac mannan gum.

5. The at least one polysaccharide as claimed in claim 4, wherein the at least one polysaccharide is carrageenans.

6. The at least one effect pigment as claimed in claim 1, comprises a particulate substrate selected from a group consisting of mica, synthetic mica, silica, glass flakes, and alumina.

7. The at least one effect pigment as claimed in claim 1, comprises a metal oxide compound selected from a group consisting of titanium dioxide, iron oxide, and chromium oxide.

8. The at least one effect pigment as claimed in claim 1, comprises a colorant compound selected from a group consisting of aluminum powder, bismuth oxychloride, borosilicates, ferric ferrocyanide, ultramarines, manganese violet, carmine, and organic pigments.

9. The humectant as claimed in claim 1 is selected from a group of polyols consisting of glycols, glycerin, and sugar alcohols.

10. The anhydrous phase as claimed in claim 1 comprises compounds selected from a group consisting of silicone compounds, oils, esters, hydrocarbons, polymers, emulsifiers, and waxes.

11. A buoyant cosmetic gel formulation comprising:
a volatile liquid, wherein the volatile liquid is an aqueous liquid with similar volatility to water;
at least one polysaccharide;
at least one effect pigment;
a humectant;
an anhydrous phase;
the volatile liquid being found ranging between 30.0 to 70.0 wt % of the formulation;
the at least one polysaccharide being found ranging between 0.01 to 10.0 wt % of the formulation;
the at least one effect pigment being found ranging between 10.0 to 50.0 wt % of the formulation;
the humectants being found ranging between 1.0 to 30.0 wt % of the formulation; and
the anhydrous phase being found ranging between 1.0 to 30.0 wt % of the formulation.

12. The at least one polysaccharide as claimed in claim 11 is selected from a group consisting of gellan gum, xanthan gum, agar, carrageenans, locust bean gum, acacia senegal gum, guar gum, and konjac mannan gum.

13. The at least one polysaccharide as claimed in claim 12, wherein the at least one polysaccharide is carrageenans.

14. The at least one effect pigment as claimed in claim 11, comprises a particulate substrate selected from a group consisting of mica, synthetic mica, silica, glass flakes, and alumina.

15. The at least one effect pigment as claimed in claim 11, comprises a metal oxide compound selected from a group consisting of titanium dioxide, iron oxide, and chromium oxide.

16. The at least one effect pigment as claimed in claim 11, comprises a colorant compound selected from a group consisting of aluminum powder, bismuth oxychloride, borosilicates, ferric ferrocyanide, ultramarines, manganese violet, carmine, and organic pigments.

17. The humectant as claimed in claim 11 is selected from a group of polyols consisting of glycols, glycerin, and sugar alcohols.

18. The anhydrous phase as claimed in claim 11 comprises compounds selected from a group consisting of silicone compounds, oils, esters, hydrocarbons, polymers, emulsifiers, and waxes.
